(19) United States
(12) Patent Application Publication
(10) Pub. No.: US 2021/0190775 A1
(43) Pub. Date: Jun. 24, 2021

(54) COMPOSITIONS AND METHODS FOR TAGGING AND DETECTING NUCLEIC ACIDS

(71) Applicant: Nanoco Technologies Ltd., Manchester (GB)

(72) Inventor: Imad Naasani, Manchester (GB)

(21) Appl. No.: 17/125,313

(22) Filed: Dec. 17, 2020

Related U.S. Application Data
(60) Provisional application No. 62/949,631, filed on Dec. 18, 2019.

(51) Int. Cl. G01N 33/543; G01N 33/58

(52) U.S. Cl. CPC G01N 33/54346; G01N 33/588; G01N 33/582

(57) ABSTRACT

Compositions of matter according to the disclosure include a nanoparticle-dye conjugate having a photoluminescent or fluorescent nanoparticle associated with a fluorescent dye. Methods of imaging a biological sample according to the disclosure include treating a biological sample with a composition of matter a nanoparticle-dye conjugate, associating the nanoparticle-dye conjugate with the biological sample, and imaging the nanoparticle-dye conjugate associated biological sample.

### CROSS REFERENCE TO RELATED APPLICATION

This application claims the benefit of U.S. Provisional Application Ser. No. 62/949,631, filed Dec. 18, 2019, which is incorporated by reference herein in its entirety.

### BACKGROUND OF THE INVENTION

### 1. Field of the Invention

The invention generally relates to the formation of quantum dot (QD)-fluorescent dye conjugates for biological diagnostics and imaging applications.

### 2. Description of the Related Art Including Information Disclosed Under 37 CFR 1.97 and 1.98

Conventional methods for labeling nucleotides are straightforward, but suffer from low sensitivity and limited versatility due to specific spectral requirements during measurement. There is constant need for maximizing the detection ability of nucleic acids.

Efforts to provide in vivo labelling and identification of tumor cells sufficient to support adequate resection has been undertaken. However, small molecule dyes, organic dyes and carbon black inks lack specificity and tend to quickly stain all surrounding tissue. Recently, fluorescence imaging using organic dyes has been introduced and, while fluorescent dyes can improve selectivity, they are limited by their rapid clearance, fast fading, fast metabolic degradation, low photostability in aqueous media, and low quantum yield. See e.g. Condeelis J and Weissleder R. In Vivo Imaging in Cancer. Cold Spring Harb Perspect Biol. 2010, 2:a003848.

### BRIEF DESCRIPTION OF THE DRAWINGS

FIG. 1 shows an exemplary reaction procedure for the formation of a QD-fluorescent dye conjugate in accordance with aspects of the disclosure.

FIG. 2 is a graph showing ultraviolet-visible (UV-Vis) absorption spectra of (1) QD nanoparticles prior to conjugation with a fluorescent dye and (2) QD nanoparticle-fluorescent dye conjugates according to various aspects of the disclosure.

FIG. 3 is a graph showing fluorescence emission spectra of (A) a fluorescent dye in the presence of DNA, (2) a QD nanoparticle-fluorescent dye conjugate in accordance with various aspects of the disclosure, and (C) the QD nanoparticle-fluorescent dye conjugate in the presence of DNA. In FIG. 3, the y-axis is provided in relative fluorescence units (RFU).

FIG. 4A is a graph showing fluorescence emission spectra of solutions containing varying concentrations of DNA and a fluorescent dye.

FIG. 4B is a graph showing fluorescence emission spectra of solutions containing varying concentrations of DNA and a QD nanoparticle-fluorescent dye conjugate.

FIG. 5 shows fluorescence microscopy images of QD nanoparticle-fluorescent dye conjugates according to various aspects of the disclosure in the presence of DNA threads mounted on a glass slide (top left and right), QD nanoparticle-fluorescent dye conjugates according to various aspects of the disclosure on a glass slide absent DNA threads (bottom left), and fluorescent dye with DNA threads but without QD nanoparticles (as a reference, bottom right).

FIG. 6 shows a zoomed in fluorescence microscopy image of QD nanoparticle-fluorescent dye conjugates according to various aspects of the disclosure in the presence of DNA threads mounted on a glass slide as in FIG. 5.

FIG. 7 shows fluorescence microscopy images of chromosomes from Mia Pa Ca-2 human pancreatic carcinoma cells treated with QD nanoparticle-fluorescent dye conjugates according to various aspects of the disclosure (left) and a fluorescent dye alone (right).

FIG. 8 shows fluorescence microscopy images of A431 human squamous cell carcinoma cells treated with QD nanoparticle-fluorescent dye conjugates according to various aspects of the disclosure (left) and a fluorescent dye alone (right).

FIG. 9 shows fluorescence microscopy images of MiaPaca2 pancreatic carcinoma treated with QD nanoparticle-fluorescent dye conjugates according to various aspects of the disclosure (left) and a QDs alone (right).

FIG. 10 shows fluorescence spectra of a fluorescent dye alone, quantum dots alone, and a QD-fluorescent dye conjugate formed from the fluorescent dye and the quantum dots according to various aspects of the disclosure.

FIG. 11 shows UV-Vis absorption spectra of a fluorescent dye alone, quantum dots alone, and a QD-fluorescent dye conjugate formed from the fluorescent dye and the quantum dots according to various aspects of the disclosure.

### DETAILED DESCRIPTION

The following description of the embodiments is merely exemplary in nature and is in no way intended to limit the subject matter of the disclosure, their application, or uses.

As used throughout, ranges are used as shorthand for describing each and every value that is within the range. Any value within the range can be selected as the terminus of the range. Unless otherwise specified, all percentages and amounts expressed herein and elsewhere in the specification should be understood to refer to percentages by weight.

For the purposes of this specification and appended claims, unless otherwise indicated, all numbers expressing quantities, percentages or proportions, and other numerical values used in the specification and claims, are to be understood as being modified in all instances by the term “about.” The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent, alternatively ±5 percent, and alternatively ±1 percent of the given numeric value provided such a deviation does not alter the end function or result of the value. Accordingly, unless indicated to the contrary, the numerical parameters set forth in this specification and attached claims are approximations that can vary depending upon the desired properties sought to be obtained by the invention.

It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. For example, as used in this specification and the following claims, the terms “comprise” (as well as forms, derivatives, or variations thereof, such as “comprising” and “comprises”), “include” (as well as forms, derivatives, or variations thereof, such as “including” and “includes”) and “has” (as well as forms, derivatives, or variations thereof, such as “having” and “have”) are inclusive (i.e., open-ended) and do not exclude additional elements or steps. Accordingly, these terms are intended to not only cover the recited element(s) or step(s), but may also include other elements or steps not expressly recited. Furthermore, as used herein, the use of the terms “a” or “an” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

As used herein, the term “nanoparticle” is used to describe a particle with dimensions on the order of approximately 1 to 100 nm. The term “quantum dot” (QD) is used to describe a semiconductor nanoparticle displaying quantum confinement effects. The dimensions of QDs are typically, but not exclusively, between 1 to 10 nm. The terms “nanoparticle” and “quantum dot” are not intended to imply any restrictions on the shape of the particle. The term “nanorod” is used to describe a prismatic nanoparticle having lateral dimensions, x and y, and length, z, wherein z>x,y.

Methods of synthesizing core and core-shell nanoparticles are disclosed, for example, in co-owned U.S. Pat. Nos. 7,867,556, 7,867,557, 7,803,423, 7,588,828, and 6,379,635. The contents of each of the forgoing patents are hereby incorporated by reference herein in their entirety. U.S. Pat. Nos. 9,115,097, 8,062,703, 7,985,446, 7,803,423, and 7,588,828, and U.S. Publication Nos. 2010/0283005, 2014/0264196, 2014/0277297 and 2014/0370690, the entire contents of each of which are hereby incorporated by reference herein, describe methods of producing large volumes of high quality monodisperse QDs.

A nanoparticle's compatibility with a medium as well as the nanoparticle's susceptibility to agglomeration, photo-oxidation and/or quenching, is mediated largely by the surface composition of the nanoparticle. The coordination about the final inorganic surface atoms in any core, core-shell or core/multi-shell nanoparticle may be incomplete, with highly reactive “dangling bonds” on the surface, which can lead to particle agglomeration. This problem may be overcome by passivating (capping) the “bare” surface atoms with protecting organic groups, referred to herein as capping ligands or a capping agent. The capping or passivating of particles prevents particle agglomeration from occurring, protects the particle from its surrounding chemical environment, and provides electronic stabilization (passivation) to the particles, in the case of core material. The capping ligand is usually a Lewis base bound to surface metal atoms of the outer most inorganic layer of the particle. The nature of the capping ligand largely determines the compatibility of the nanoparticle with a particular medium.

In many QD materials, the capping ligands are hydrophobic (for example, alkyl thiols, fatty acids, alkyl phosphines, alkyl phosphine oxides, and the like). Thus, the nanoparticles are typically dispersed in hydrophobic solvents, such as toluene, following synthesis and isolation of the nanoparticles. Such capped nanoparticles are typically not dispersible in more polar media. If surface modification of the QD is desired, the most widely used procedure is known as ligand exchange, where lipophilic ligand molecules that coordinate to the surface of the nanoparticle during core synthesis and/or shelling procedures may subsequently be exchanged with a polar/charged ligand compound. An alternative surface modification strategy intercalates polar/charged molecules or polymer molecules with the ligand molecules that are already coordinated to the surface of the nanoparticle. However, while certain ligand exchange and intercalation procedures render the nanoparticle more compatible with aqueous media, they may result in materials of lower photoluminescence quantum yield (QY) and/or substantially larger size than the corresponding unmodified nanoparticle. Problematically, for the theranostic purposes disclosed herein, the QD is preferably substantially free of toxic heavy metals such as cadmium, lead and arsenic (e.g., contains less than 5 wt. %, such as less than 4 wt. %, less than 3 wt. %, less than 2 wt. %, less than 1 wt. %, less than 0.5 wt. %, less than 0.1 wt. %, less than 0.05 wt. %, or less than 0.01 wt. % of heavy metals such as cadmium, lead and arsenic) or is free of heavy metals such as cadmium, lead and arsenic. Examples of cadmium-, lead- and arsenic-free nanoparticles include nanoparticles comprising semiconductor materials, e.g., ZnS, ZnSe, ZnTe, InP, InAs, InSb, AlP, AlS, AlAs, AlSb, GaN, GaP, GaAs, GaSb, AgInS₂, AgInS₂/ZnS, Si, Ge, and alloys and doped derivatives thereof, particularly, nanoparticles comprising cores of one of these materials and one or more shells of another of these materials. In some circumstances, however, the use of QDs that contain toxic elements like Cd, As, Hg, or Pb is warranted for research purposes or for otherwise medically allowed doses.

It is noted that nanoparticles that include a single semiconductor material, e.g., ZnS, ZnSe, InP, GaN, etc. may have relatively low QY because of non-radiative electron-hole recombination that occurs at defects and dangling bonds at the surface of the nanoparticles. In order to at least partially address these issues, the nanoparticle cores may be at least partially coated with one or more layers (also referred to herein as “shells”) of a material different than that of the core, for example a different semiconductor material than that of the “core.” The material included in the one or more shells may incorporate ions from any one or more of groups 2 to 16 of the periodic table. When a nanoparticle has two or more shells, each shell may be formed of a different material. In an exemplary core/shell QD material, the core is formed from one of the materials specified above and the shell includes a semiconductor material of larger band-gap energy and similar lattice dimensions as the core material. Exemplary shell materials include, but are not limited to, ZnS, ZnO, MgS, MgSe, MgTe and GaN. One example of a multi-shell QD nanoparticle is InP/ZnS/ZnO. The confinement of charge carriers within the core and away from surface states provides nanoparticles of greater stability and higher QY.

However, while it is desirable to have QD that lack toxic heavy metals, it has proved particularly difficult to modify the surface of cadmium-free QDs. Cadmium-free QDs readily degrade when methods such as ligand exchange are used to modify the surface of such cadmium-free QDs. For example, attempts to modify the surface of cadmium-free QDs have been observed to cause a significant decrease in the QY of such nanoparticles. For the in vivo purposes disclosed herein, surface-modified cadmium-free QDs with high QY are required. For purposes of the invention, when referring to water dispersible cadmium-free QDs: QY of <20% are considered very low; QY of <30% are considered low; QY of 30-40% are considered medium; QY >40% are considered high and QY >50% are considered very high.

The high QY cadmium-free water dispersible QDs disclosed herein have a QY greater than about 20%. For certain in vivo embodiments, heavy metal-free semiconductor indium-based QDs or QDs containing indium and/or phosphorus are preferred.

QDs used in accordance with varying aspects of the disclosure can have a size ranging from 1-15 nm before surface functionalization. In some instances, the QDs can be core QDs. In some instances, the QDs can be core-shell QDs. In some instances, the QDs can be core-multishell QDs. QDs used in accordance with various aspects of the disclosure can be made of, or include a core material comprising:

IIA-VIA (2-16) material, consisting of a first element from group 2 of the periodic table and a second element from group 16 of the periodic table and also including ternary and quaternary materials and doped materials. IIA-VIA nanoparticle material includes but is not restricted to: MgS, MgSe, MgTe, CaS, CaSe, CaTe, SrS, SrSe, SrTe, BaS, BaSe, BaTe;

IIB-VIA (12-16) material consisting of a first element from group 12 of the periodic table and a second element from group 16 of the periodic table and also including ternary and quaternary materials and doped materials. IIB-VIA nanoparticle material includes but is not restricted to: ZnS, ZnSe, ZnTe, CdS, CdSe, CdTe, HgS, HgSe, HgTe;

II-V material, consisting of a first element from group 12 of the periodic table and a second element from group 15 of the periodic table and also including ternary and quaternary materials and doped materials. II-V nanoparticle material includes but is not restricted to: Zn₃P₂, Zn₃N₂, Zn₃As₂, Cd₃P₂, Cd₃N₂, Cd₃As₂;

III-V material, consisting of a first element from group 13 of the periodic table and a second element from group 15 of the periodic table and also including ternary and quaternary materials and doped materials. III-V nanoparticle material includes but is not restricted to: BP, AlAs, AlN, AlP, AlSb, GaAs, GaN, GaP, GaSb; InAs, InN, InP, InSb, BN;

III-IV material, consisting of a first element from group 13 of the periodic table and a second element from group 14 of the periodic table and also including ternary and quaternary materials and doped materials. III-IV nanoparticle material includes but is not restricted to: B₄C, Al₄C₃, Ga₄C;

III-VI material, consisting of a first element from group 13 of the periodic table and a second element from group 16 of the periodic table and also including ternary and quaternary materials. III-VI nanoparticle material includes but is not restricted to: Al₂S₃, Al₂Se₃, Al₂Te₃, Ga₂S₃, Ga₂Se₃, GeTe; In₂S₃, In₂Se₃, Ga₂Te₃, In₂Te₃, InTe;

IV-VI material, consisting of a first element from group 14 of the periodic table and a second element from group 16 of the periodic table, and also including ternary and quaternary materials and doped materials. IV-VI nanoparticle material includes but is not restricted to: PbS, PbSe, PbTe, SnS, SnSe, SnTe;

V-VI material, consisting of a first element from group 15 of the periodic table and a second element from group 16 of the periodic table, and also including ternary and quaternary materials and doped materials. V-VI nanoparticle material includes but is not restricted to: Bi₂Te₃, Bi₂Se₃, Sb₂Se₃, Sb₂Te₃; and

Nanoparticle material, consisting of a first element from any group in the transition metal of the periodic table, and a second element from group 16 of the periodic table and also including ternary and quaternary materials and doped materials. Nanoparticle material includes but is not restricted to: NiS, CrS, CuInS₂, AgInS₂.

By the term doped nanoparticle for the purposes of specifications and claims, refers to nanoparticles of the above and a dopant comprised of one or more main group or rare earth elements, this most often is a transition metal or rare earth element, such as but not limited to ZnS or InP nanoparticles doped with Mn²⁺, Ca²⁺, Mg²⁺, and Al³⁺.

The term “ternary material,” for the purposes of specifications and claims, refers to QDs of the above but a three-component material. The three components are usually compositions of elements from the as mentioned groups Example being (InₓGa₁₋ₓP)ₘLₙ nanocrystal (where L is a capping agent).

The term “quaternary material,” for the purposes of specifications and claims, refers to nanoparticles of the above but a four-component material. The four components are usually compositions of elements from the as mentioned groups Example being (InPZnS)ₘLₙ nanocrystal (where L is a capping agent).

The material used on any shell or subsequent numbers of shells grown onto the core particle in most cases will be of a similar lattice type material to the core material, i.e. have close lattice match to the core material so that it can be epitaxially grown on to the core, but is not necessarily restricted to materials of this compatibility. The material used on any shell or subsequent numbers of shells grown on to the core present in most cases will have a wider bandgap than the core material but is not necessarily restricted to materials of this compatibility. The materials of any shell or subsequent numbers of shells grown on to the core can include material comprising:

IIA-VIA (2-16) material, consisting of a first element from group 2 of the periodic table and a second element from group 16 of the periodic table and also including ternary and quaternary materials and doped materials. IIA-VIA shell material includes but is not restricted to: MgS, MgSe, MgTe, CaS, CaSe, CaTe, SrS, SrSe, SrTe;

IIB-VIA (12-16) material, consisting of a first element from group 12 of the periodic table and a second element from group 16 of the periodic table and also including ternary and quaternary materials and doped materials. IIB-VIA shell material includes but is not restricted to: ZnS, ZnSe, ZnTe, CdS, CdSe, CdTe, HgS, HgSe, HgTe;

II-V material, consisting of a first element from group 12 of the periodic table and a second element from group 15 of the periodic table and also including ternary and quaternary materials and doped materials. II-V shell material includes but is not restricted to: Zn₃P₂, Zn₃N₂, Zn₃As₂, Cd₃P₂, Cd₃N₂, Cd₃As₂;

III-V material, consisting of a first element from group 13 of the periodic table and a second element from group 15 of the periodic table and also including ternary and quaternary materials and doped materials. III-V shell material includes but is not restricted to: BP, AlAs, AlN, AlP, AlSb; GaAs, GaN, GaP, GaSb, InAs, InN, InP, InSb, BN;

III-IV material, consisting of a first element from group 13 of the periodic table and a second element from group 14 of the periodic table and also including ternary and quaternary materials and doped materials. III-IV shell material includes but is not restricted to: B₄C, Al₄C₃, Ga₄C;

III-VI material, consisting of a first element from group 13 of the periodic table and a second element from group 16 of the periodic table and also including ternary and quaternary materials. III-VI shell material includes but is not restricted to: Al₂S₃, Al₂Se₃, Al₂Te₃, Ga₂S₃, Ga₂Se₃, In₂S₃, In₂Se₃, Ga₂Te₃, In₂Te₃;

IV-VI material, consisting of a first element from group 14 of the periodic table and a second element from group 16 of the periodic table and also including ternary and quaternary materials and doped materials. IV-VI shell material includes but is not restricted to: PbS, PbSe, PbTe, SnS, SnSe, SnTe;

V-VI material, consisting of a first element from group 15 of the periodic table and a second element from group 16 of the periodic table, and also including ternary and quaternary materials and doped materials. V-VI shell material includes but is not restricted to: Bi₂Te₃, Bi₂Se₃, Sb₂Se₃, Sb₂Te₃; and

Nanoparticle shell material, consisting of a first element from any group in the transition metal of the periodic table, and a second element from group 16 of the periodic table and also including ternary and quaternary materials and doped materials. Nanoparticle material includes but is not restricted to: NiS, CrS, CuInS₂, AgInS₂.

In certain embodiments, non-toxic QD nanoparticles are surface modified to enable them to be water-soluble and to have surface moieties that allow derivatization by exposing them to a ligand interactive agent to effect the association of the ligand interactive agent and the surface of the QD. The ligand interactive agent can comprise a chain portion and a functional group having a specific affinity for, or reactivity with, a fluorescent dye, as described below. The chain portion may be, for example, an alkane chain. Examples of functional groups having a specific affinity for, or reactivity with, a fluorescent dye, include nucleophiles such as thiol groups, hydroxyl groups, carboxamide groups, ester groups, and a carboxyl groups. The ligand interactive agent may, or may not, also comprise a moiety having an affinity for the surface of a QD. Examples of moieties having an affinity for the surface of a QD include thiols, dithiocarbonates, dithiocarbamates, amines, carboxylic groups, phosphines, and phosphonic acids. If ligand interactive group does not comprise such a moiety, the ligand interactive group can associate with the surface of nanoparticle by intercalating with capping ligands. Examples of ligand interactive agents include C₈₋₂₀ fatty acids and esters thereof, such as for example myristic acid and isopropyl myristate. In accordance with various embodiments of the disclosure, at least one functional group is located at and end of the ligand interactive agent away from the QD surface such that the functional group is available for covalently or non-covalently, or chemically or physically, binding with a fluorescent dye.

It should be noted that the ligand interactive agent may be associated with a QD nanoparticle simply as a result of the processes used for the synthesis of the nanoparticle, obviating the need to expose nanoparticle to additional amounts of ligand interactive agents. In such case, there may be no need to associate further ligand interactive agents with the nanoparticle. Alternatively, or in addition, a QD nanoparticle may be exposed to ligand interactive agent after the nanoparticle is synthesized and isolated. For example, the nanoparticle may be incubated in a solution containing the ligand interactive agent for a period of time. Such incubation, or a portion of the incubation period, may be at an elevated temperature to facilitate association of the ligand interactive agent with the surface of the nanoparticle. Following association of the ligand interactive agent with the surface of nanoparticle, the QD nanoparticle is exposed to a fluorescent dye such that the QD nanoparticle and fluorescent dye associate with each other to form a QD nanoparticle-fluorescent dye conjugate (alternatively referred to herein as a QD-fluorescent dye conjugate or a QD-dye conjugate). Fluorescent dyes used in accordance with various aspects of the disclosure include one or more functional groups which may bind with a functional group of the ligand interactive agent, forming the QD-fluorescent dye conjugate. In some instances, the one or more functional groups of the fluorescent dye and the functional group of the ligand interactive agent are covalently bound to each other. In some instances, the one or more functional groups of the fluorescent dye and the functional group of the ligand interactive agent are non-covalently bound to each other. In some instances, the one or more functional groups of the fluorescent dye and the functional group of the ligand interactive agent are chemically bound to each other. In some instances, the one or more functional groups of the fluorescent dye and the functional group of the ligand interactive agent are physically bound to each other.

In certain embodiments of the disclosure the QD of the QD-fluorescent dye conjugate can be a core, core/shell or core/multi-shell QD as described above. In certain embodiments, the QD of the QD-fluorescent dye conjugate can be a photoluminescent or fluorescent nanoparticle other than a QD as described above. Examples of photoluminescent or fluorescent nanoparticles that me be used include, but are not limited to, nanodiamonds, fluorescent silica nanobeads, fluorescent polymer nanoparticles, fluorescent elemental metal nanoparticles, rare earth-doped nanoparticles, graphene quantum dots (GQDs), carbon quantum dots (CQDs), and perovskite quantum dots.

In certain embodiments, nanodiamond-fluorescent dye conjugates can be prepared for biological diagnostics and imaging applications. Nanodiamonds in accordance with the disclosure may have diameters ranging from about 1 to about 140 nm, preferably about 5 to about 35 nm, as determined by dynamic light scattering (DLS). Carboxylate functionalized (for example, Sigma Aldrich, catalog Nos. 900172, 900177, 900184), hydroxyl functionalized (for example, Sigma Aldrich, catalog No. 900174, 900179), dodecane functionalized (for example, Sigma Aldrich, catalog No. 901967), octadecane functionalized (for example, Sigma Aldrich, catalog No. 901770), amine functionalized (for example, Sigma Aldrich, catalog No. 901799), and PEG coated (for example, Sigma Aldrich, catalog Nos. 901798, 901800, 901802, 901803) nanodiamonds are commercially available.

In certain embodiments, fluorescent silica nanobead-fluorescent dye conjugates can be prepared for biological diagnostics and imaging applications. Fluorescent silica nanobeads in accordance with the disclosure may have diameters ranging from about 25 to about 120 nm and are commercially available (for example, Sigma Aldrich, catalog Nos. 797936, 797928, 797898, 797863, 797952, 797871, 797944, 797901).

In certain embodiments, fluorescent polymer nanoparticle-fluorescent dye conjugates can be prepared for biological diagnostics and imaging applications. Fluorescent polymer nanoparticles in accordance with the disclosure include, but are not limited to green fluorescent poly(D,L-lactide-co-glycolide) (PLGA) nanoparticles (for example, 100-500 nm average diameters; Sigma Aldrich, Catalog Nos. 805157, 805211, 805300); carboxyl-functionalized purple-, blue-, green- and red-fluorescent polystyrene and crosslinked poly(styrene/divinylbenzene) microspheres (for example, Bangs Laboratories, Inc., catalog Nos. FCDG001 through FCDG009, FCFR001 through FCFR006, FCSG003, FCGB003, FCGB006, FCGB008, FCSY006, FCSY007, FCEG006, FCEG008), carboxylate-modified polystyrene latex beads (for example, Sigma Aldrich, catalog No. L5155), sulfate-modified polystyrene latex beads (for example, Sigma Aldrich, catalog No. L1528, L9902), and amine-modified polystyrene latex beads (for example, Sigma Aldrich, catalog No. L9904). In certain embodiments, metals or metal oxides, such as iron oxide, can be incorporated into the polymer nanoparticles (for example, Sigma Aldrich, Catalog Nos. 905054, 905038, 904996, 905046; and Bangs Laboratories, Inc., catalog Nos. MCDG001, MCFR001, MEDG001, MEFR001, MEDG002, MESY002, MEGB002, MEFR002, UMGB001, UMDG001, UMEG001, UMFR001, UMGB002, UMDG002, UMEG002, UMFR002, UMGB003, UMDG003, UMEG003, UMFR003).

In some instances, fluorescent elemental metal nanoparticle-fluorescent dye conjugates can be prepared for biological diagnostics and imaging applications. Fluorescent elemental metal nanoparticles in accordance with the disclosure include, but are not limited to gold nanorods or nanospheres functionalized with biotin, amines, NHS, streptavidin, azides, maleimide, alkyl chains, or carboxylates, all of which are commercially available from Sigma Aldrich or American Elements, (1-mercaptoundec-11-yl)tetra(ethylene glycol)-functionalized gold nanoparticles (for example, American Elements, product codes, AU-H2O-02-FNPD, AU-H2O-03-FNPD, AU-H2O-04-FNPD, AU-H2O-05-FNPD), carboxylate-functionalized silver nanoparticles (for example, American Elements, product code AG-M-01-NPD.COOHF).

In some instances, rare earth doped nanoparticle-fluorescent dye conjugates can be prepared for biological diagnostics and imaging applications. Rare earth-doped nanoparticle in accordance with the disclosure include, but are not limited to Y(P,V)O₄:Eu nanoparticles (for example, Sigma Aldrich, catalog No. 900557, 10 nm diameters, fluorescence λₑₘ 620 nm, ethylene glycol functionalized), LaPO₄:Ce,Tb nanoparticles (for example, Sigma Aldrich, catalog No. 900558, 15 nm diameters, fluorescence λₑₘ 545 nm, ethylene glycol functionalized), BaSO₄:Eu nanoparticles (for example, Sigma Aldrich, catalog No. 900559, fluorescence λₑₘ 390 nm), Y(V,P)O₄:Bi,Dy nanoparticles (for example, Sigma Aldrich, catalog No. 900591, 6 nm diameters, fluorescence λₑₘ 580 nm, ethylene glycol functionalized), NaYF₄:Yb nanoparticles (for example, Sigma Aldrich, catalog No. 900544, 17 nm diameters, fluorescence λₑₘ 474 nm, oleic acid functionalized, or catalog No. 900556, 20 nm diameters, fluorescence λₑₘ 540 nm, oleic acid functionalized).

In some instances, graphene quantum dot (GQD)-fluorescent dye conjugates can be prepared for biological diagnostics and imaging applications. GQDs in accordance with the disclosure include, but are not limited to green luminescent GQDs (for example, Sigma Aldrich, catalog No. 900712 fluorescence λₑₘ 520-540 nm), and blue luminescent GQDs (for example, Sigma Aldrich, catalog No. 900708, fluorescence λₑₘ 435-450 nm).

In some instances, carbon quantum dot (CQD)-fluorescent dye conjugates can be prepared for biological diagnostics and imaging applications. CQDs in accordance with the disclosure may have diameters ranging from about 1 to about 20 nm, preferably about 1.5 to about 3 nm, as determined by dynamic light scattering (DLS). Water-dispersed CQDs (for example, Sigma Aldrich, catalog No. 900414, fluorescence λₑₘ 450-550 nm), are commercially available for utilization in this invention.

In some instances, perovskite quantum dot-fluorescent dye conjugates can be prepared for biological diagnostics and imaging applications. Perovskite quantum dots in accordance with the disclosure may have diameters ranging from about 1 to about 20 nm, preferably about 4 to about 15 nm, as determined by dynamic light scattering (DLS). Water dispersed perovskite quantum dots can be prepared using ligand exchange or by a process as described in Example 2 below. For this purpose, several commercial sources of perovskite quantum dots dispersed in organic solvents or oils can be used (for example, Sigma Aldrich, catalog Nos. 900746, 900747, 900748, 905062, fluorescence λₑₘ 450-530 nm).

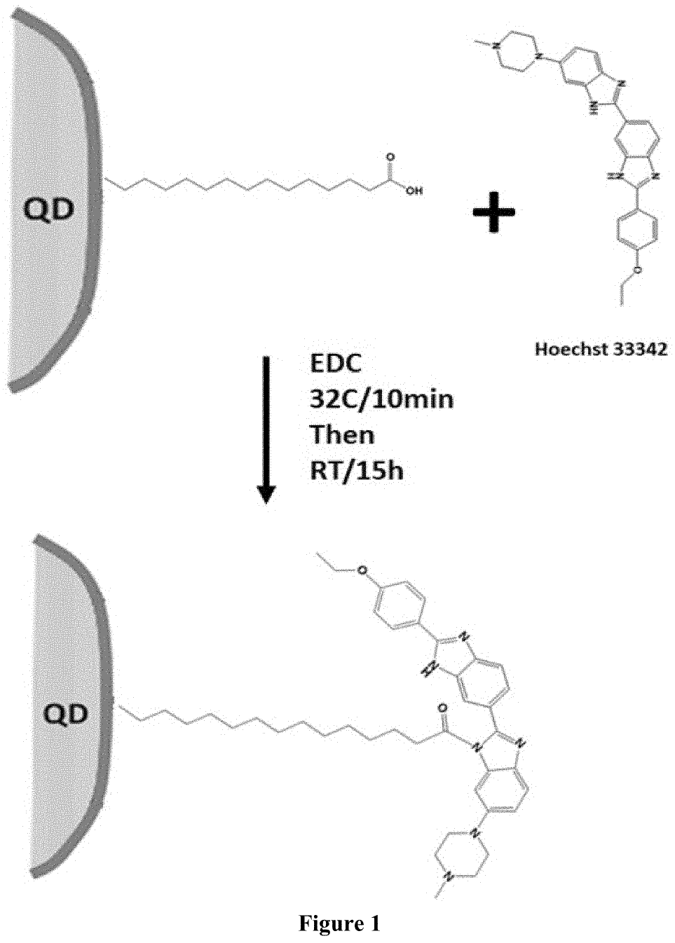

In some embodiments according to the disclosure, the fluorescent dye is Hoechst 33342. Hoechst 33342 is a fluorescent bisbenzimide derivative (2′-(4-ethoxyphenyl)-6-(4-methyl-1-piperazinyl)-1H,3′H-2,5′-bibenzimidazole) that can bind to nucleic acids, particularly the adenine-thymine-rich regions in DNA. It has an absorption peak at 354 nm and emission peak at 486 nm. As discussed below, QD-Hoechst 3342 conjugates exhibit a significant enhancement in the fluorescence intensity of Hoechst 33342 and an ability of nanoparticle vectorization to the nuclei of live or fixed cells in cell cultures or tissues. This is significant as all nanoparticles are normally transferred into cytoplasmic vesicles like the endosomes after uptake by the cells. An exemplary methodology for the formation of QD-Hoechst 33342 conjugates, using 1-ethyl-3-(3-dimethylaminopropyl)carbodiimide (EDC) as a coupling agent, is shown in FIG. 1.

In some instances, suitable fluorescent dyes for the fabrication of QD-fluorescent dye conjugates include, but are not limited to, the following compounds or any suitable derivatives or analogs thereof:

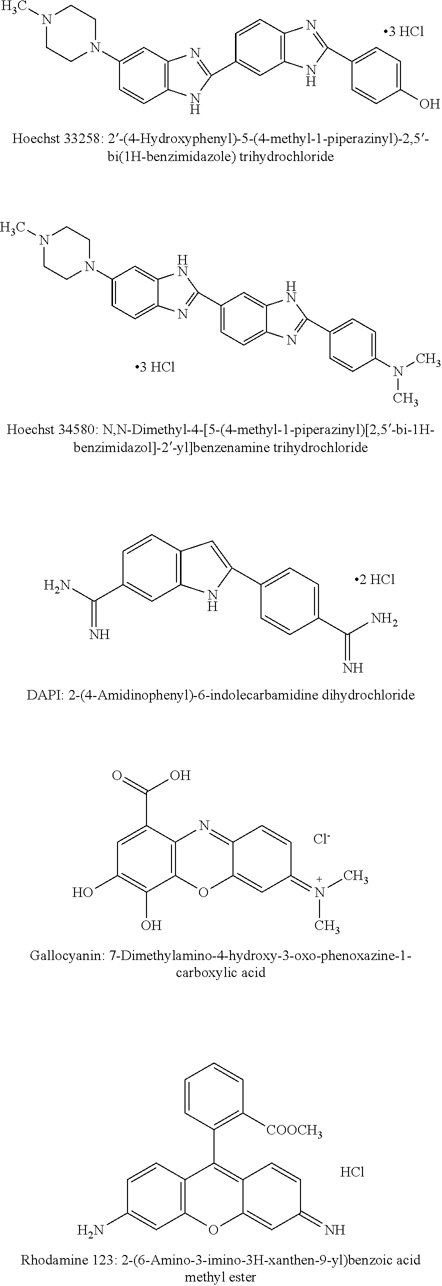

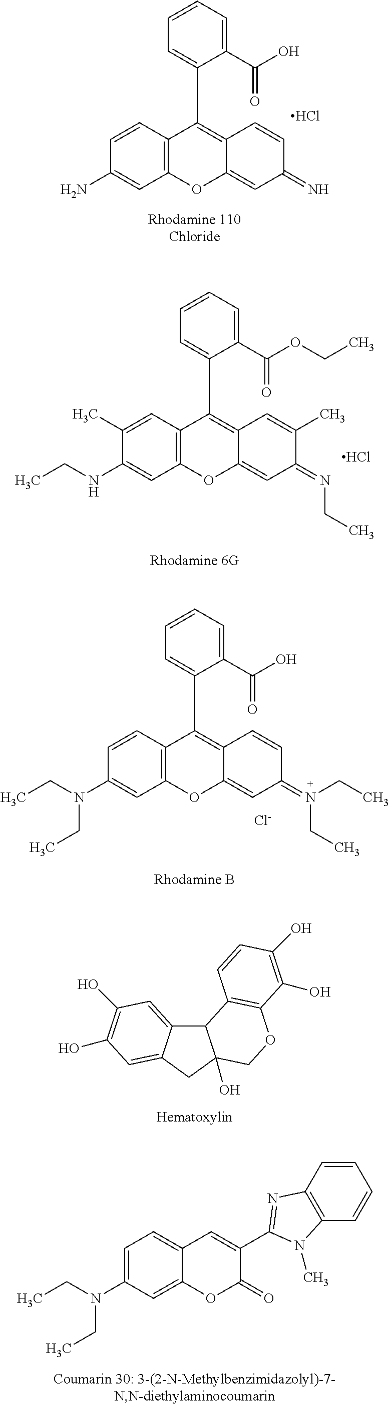

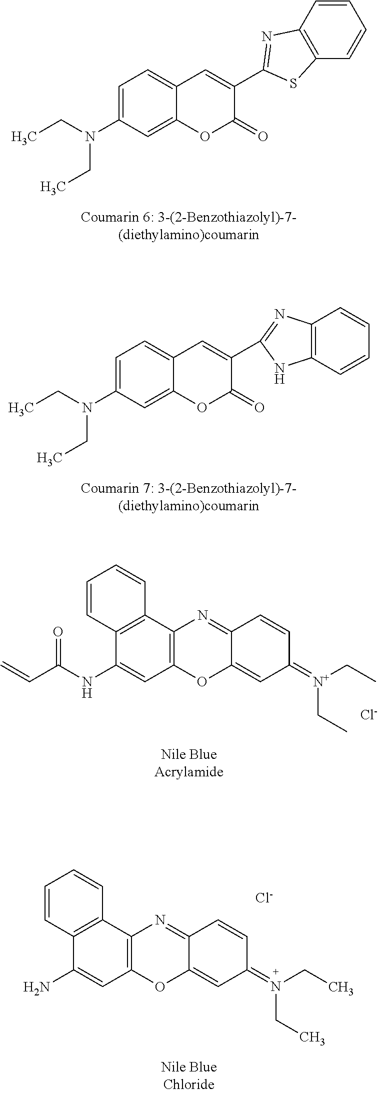

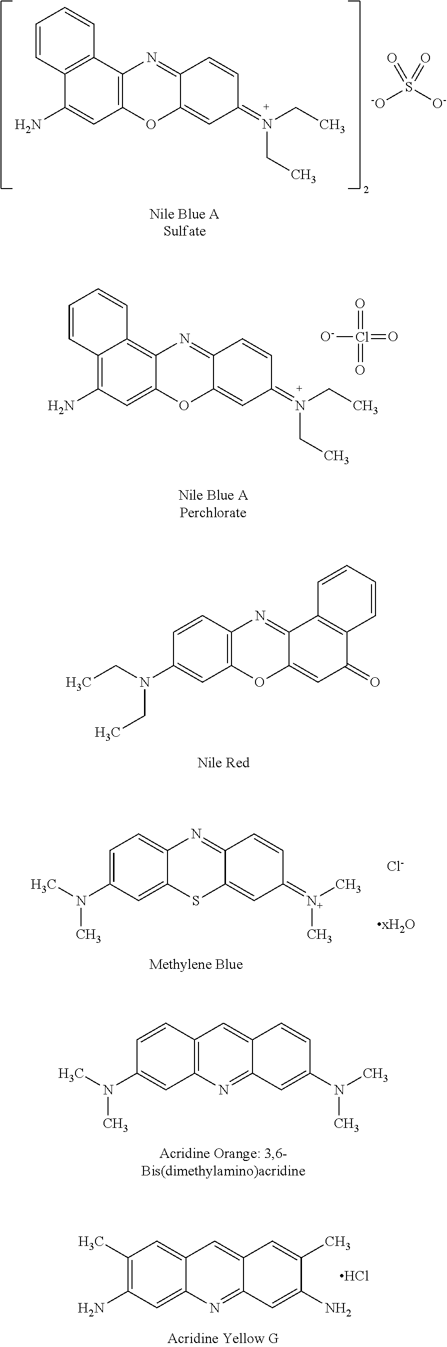

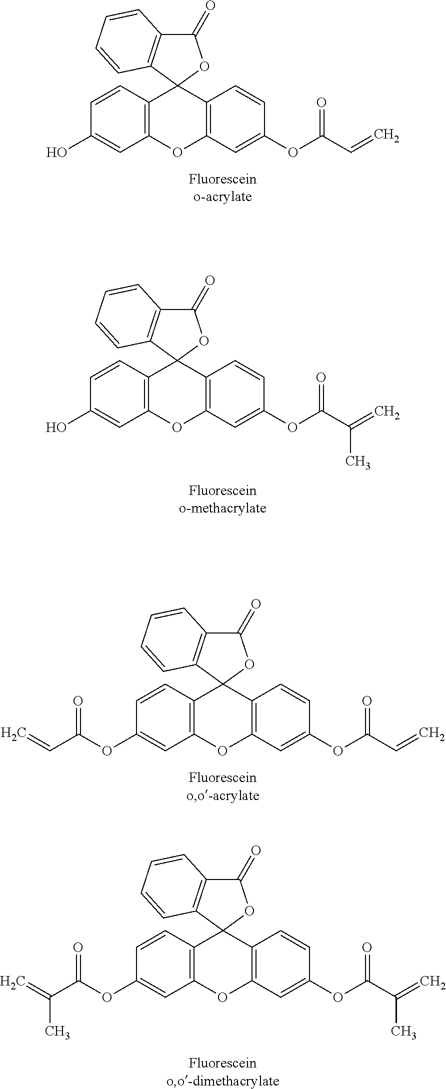

QD-fluorescent dye conjugates according to the disclosure have been found to exhibit significantly enhanced emission compared to corresponding fluorescent dyes alone. Furthermore, QD-fluorescent dye conjugates according to the disclosure exhibit, when excited with a suitable excitation source (normal or multi-photon), two emission peaks, one from the quantum dot and the other from the associated dye.

QD-fluorescent dye conjugates according to the disclosure are able to bind or intercalate with DNA or RNA types of nucleic acids. QD-fluorescent dye conjugates according to the disclosure are also able to bind or intercalate particularly to dsDNA types of nucleic acids. QD-fluorescent dye conjugates according to the disclosure have also been found to exhibit fluorescence enhancement when associated with a nucleic acid. QD-fluorescent dye conjugates according to the disclosure may also be useful to target the nuclei of eukaryotes and the nucleic acid matter of prokaryotes in live and fixed cells or organisms. QD-fluorescent dye conjugates according to the disclosure may also be useful as a gene delivery system that can carry a payload of nucleic acids (oligonucleotides, plasmids, RNAi, CRISPRi). QD-fluorescent dye conjugates according to the disclosure may also useful for the labelling of nucleic acids and oligonucleotides in vivo and in vitro. QD-fluorescent dye conjugates according to the disclosure may also be useful for the banding of chromosomes. QD-fluorescent dye conjugates according to the disclosure may also be useful for the determination of sex ratio of spermatozoa to separate X and Y-bearing sperms. QD-fluorescent dye conjugates according to the disclosure may also be useful for cell labelling in fluorescence activated cell sorting (FACS) machines. QD-fluorescent dye conjugates according to the disclosure have also been found very useful in the labelling and imaging of various forms of carcinoma cells.

A method of imaging a biological sample, the method includes treating a biological sample with a QD-fluorescent dye conjugate according to various aspect of the disclosure, associating the nanoparticle-dye conjugate with the biological sample; and imaging the nanoparticle-dye conjugate associated biological sample. The biological sample can be, for example, nucleic acids such as DNA or RNA, oligonucleotides, nuclei of eukaryotes and the nucleic acid matter of prokaryotes in live and fixed cells or organisms, cells such as carcinoma cells, and chromosomes. In some instances, associating the nanoparticle-dye conjugate with the biological sample comprises binding the nanoparticle-dye conjugate with the biological sample. In some instances, associating the nanoparticle-dye conjugate with the biological sample comprises accumulating the nanoparticle-dye conjugate with the biological sample. In some instances, imaging the nanoparticle-dye conjugate associated biological sample comprises fluorescence microscopy. In some instances, imaging biological samples can be performed in vivo. In some instances, imaging biological samples can be performed in vitro.

The following examples are include for the sake of completeness of disclosure and to illustrate the methods of making the compositions and composites of the invention as well as to present certain characteristics of the compositions. In no way are these examples intended to limit the scope or teaching of this disclosure.

### EXAMPLES

### Example 1: Synthesis of Non-Toxic Quantum Dots

A molecular seeding process was used to generate non-toxic QDs. Briefly, the preparation of non-functionalized indium-based quantum dots with emission in the range of 500-700 nm was carried out as follows: Dibutyl ester (approximately 100 ml) and myristic acid (MA) (10.06 g) were placed in a three-neck flask and degassed at ˜70° C. under vacuum for 1 h. After this period, nitrogen was introduced and the temperature was increased to ˜90° C. Approximately 4.7 g of a ZnS molecular cluster [Et₃NH]₄[Zn₁₀S₄(SPh)₁₆] was added, and the mixture was stirred for approximately 45 min. The temperature was then increased to ˜100° C., followed by the drop-wise additions of In(MA)₃ (1M, 15 ml) followed by trimethylsilyl phosphine (TMS)₃P (1M, 15 ml). The reaction mixture was stirred while the temperature was increased to ˜140° C. At 140° C., further drop-wise additions of In(MA)₃ dissolved in di-n-butylsebacate ester (1M, 35 ml) (left to stir for 5 min) and (TMS)₃P dissolved in di-n-butylsebacate ester (1M, 35 ml) were made. The temperature was then slowly increased to 180° C., and further dropwise additions of In(MA)₃ (1M, 55 ml) followed by (TMS)₃P (1M, 40 ml) were made. By addition of the precursor in this manner, indium-based particles with an emission maximum gradually increasing from 500 nm to 720 nm were formed. The reaction was stopped when the desired emission maximum was obtained and left to stir at the reaction temperature for half an hour. After this period, the mixture was left to anneal for up to approximately 4 days (at a temperature ˜20-40° C. below that of the reaction). A UV lamp was also used at this stage to aid in annealing.

The particles were isolated by the addition of dried degassed methanol (approximately 200 ml) via cannula techniques. The precipitate was allowed to settle and then methanol was removed via cannula with the aid of a filter stick. Dried degassed chloroform (approximately 10 ml) was added to wash the solid. The solid was left to dry under vacuum for 1 day. This procedure resulted in the formation of indium-based nanoparticles on ZnS molecular clusters. In further treatments, the quantum yields of the resulting indium-based nanoparticles were further increased by washing in dilute hydrofluoric acid (HF). The quantum efficiencies of the indium-based core material ranged from approximately 25%-50%. This composition is considered an alloy structure comprising In, P, Zn and S.

Growth of a ZnS shell: A 20 ml portion of the HF-etched indium-based core particles was dried in a three-neck flask. 1.3 g of myristic acid and 20 ml di-n-butyl sebacate ester were added and degassed for 30 min. The solution was heated to 200° C., and 2 ml of 1 M (TMS)₂S was added drop-wise (at a rate of 7.93 ml/h). After this addition was complete, the solution was left to stand for 2 min, and then 1.2 g of anhydrous zinc acetate was added. The solution was kept at 200° C. for 1 hr. and then cooled to room temperature. The resulting particles were isolated by adding 40 ml of anhydrous degassed methanol and centrifuging. The supernatant liquid was discarded, and 30 ml of anhydrous degassed hexane was added to the remaining solid. The solution was allowed to settle for 5 h and then centrifuged again. The supernatant liquid was collected and the remaining solid was discarded. The QYs of the final non-functionalized indium-based nanoparticle material ranged from approximately 60%-90% in organic solvents.

### Example 2: Synthesis of Water-Soluble Surface Modified QDs

Provided herein is one embodiment of a method for generating and using melamine hexamethoxymethylmelamine (HMMM) modified fluorescent nanoparticles as drug delivery vehicles. The unique melamine-based coating presents excellent biocompatibility, low toxicity and very low non-specific binding. These unique features allow a wide range of biomedical applications both in vitro and in vivo.

One example of preparation of a suitable water-soluble nanoparticle is provided as follows: 200 mg of cadmium-free QDs with red emission at 608 nm having as a core material an alloy comprising indium and phosphorus with Zn-containing shells as described in Example 1 was dispersed in toluene (1 ml) with isopropyl myristate (100 microliters). The isopropyl myristate is included as the ligand interactive agent. The mixture was heated at 50° C. for about 1-2 minutes then slowly shaken for 15 hours at room temperature. A toluene solution (4 ml) of HMMM (CYMEL 303, available from Cytec Industries, Inc., West Paterson, N.J.) (400 mg), monomethoxy polyethylene oxide (CH₃O-PEG₂₀₀₀-OH) (400 mg), and salicylic acid (50 mg) was added to the nanoparticle dispersion. The salicylic acid that is included in the functionalization reaction plays three roles: as a catalyst, a crosslinker, and a source for reactive —COOH groups. Due in part to the preference of HMMM for —OH groups, many —COOH groups provided by the salicylic acid remain available on the QD after crosslinking.

HMMM is a melamine-based linking/crosslinking agent having the following structure:

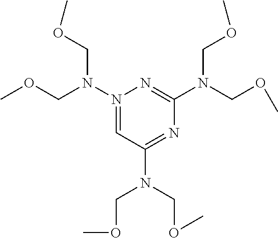

HMMM can react in an acid-catalyzed reaction to crosslink various functional groups, such as amides, carboxyl groups, hydroxyl groups, and thiols.

The mixture was degassed and refluxed at 130° C. for the first hour followed by 140° C. for 3 hours while stirring at 300 rpm with a magnetic stirrer. During the first hour a stream of nitrogen was passed through the flask to ensure the removal of volatile byproducts generated by the reaction of HMMM with nucleophiles. The mixture was allowed to cool to room temperature and stored under inert gas. The surface-modified nanoparticles showed little or no loss in fluorescence quantum yield (QY) and no change in the emission peak or full-width at half-maximum (FWHM) value, compared to unmodified nanoparticles. An aliquot of the surface-modified nanoparticles was dried under vacuum and deionized water was added to the residue. The surface-modified nanoparticles dispersed well in the aqueous media and remained dispersed permanently. In contrast, unmodified nanoparticles could not be suspended in the aqueous medium. The fluorescence QY of the surface-modified nanoparticles according to the above procedure is 40-50%. In typical batches, a quantum yield of 47%±5% is obtained.

In another embodiment, cadmium-free QDs (200 mg) with red emission at 608 nm were dispersed in toluene (1 ml) with cholesterol (71.5 mg). The mixture was heated at 50° C. for about 1-2 minutes then slowly shaken for 15 hours at room temperature. A toluene solution (4 ml) of HMMM (Cymel 303) (400 mg), monomethoxy polyethylene oxide (CH₃O-PEG₂₀₀₀-OH) (400 mg), guaifenesin (100 mg), dichloromethane (DCM) (2 mL) and salicylic acid (50 mg) was added to the nanoparticle dispersion.

As used herein the compound “guaifenesin” has the following chemical structure:

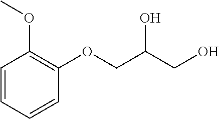

As used herein the compound “salicylic acid” has the following chemical structure:

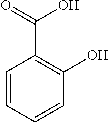

The mixture was degassed and refluxed at 140° C. for 4 hours while stirring at 300 rpm with a magnetic stirrer. As with the prior procedure, during the first hour a stream of nitrogen was passed through the flask to ensure the removal of volatile byproducts generated by the reaction of HMMM with nucleophiles. The mixture was allowed to cool to room temperature and stored under inert gas. An aliquot of the surface-modified nanoparticles was dried under vacuum and deionized water was added to the residue. The pH of the solution was adjusted to 6.5 using a 100 mM KOH solution and the excess non reacted material was removed by three cycles of ultrafiltration using Amicon filters (30 kD). The final aqueous solution was kept refrigerated until use.

It is noteworthy that traditional methods for modifying nanoparticles to increase their water solubility (e.g., ligand exchange with mercapto-functionalized water-soluble ligands) are ineffective under mild conditions to render the nanoparticles water-soluble. Under harsher conditions, such as heat and sonication, the fraction that becomes water-soluble has very low QY (<20%). The instant method, in contrast, provides water-soluble nanoparticles with high quantum yield. As defined herein, a high quantum yield is equal to or greater than 40%. In certain embodiments, a high quantum yield is obtained of equal to or greater than 45%. The surface-modified nanoparticles prepared as in this example also disperse well and remain permanently dispersed in other polar solvents, including ethanol, propanol, acetone, methylethylketone, butanol, tripropylmethylmethacrylate, or methylmethacrylate.

### Example 3: Preparation of QD-Fluorescent Dye Conjugate Nanoparticles

Water-soluble surface modified quantum dot nanoparticles (5 mg), prepared substantially as described in Example 2 were dispersed in (4-(2-hydroxyethyl)-1-piperazineethanesulfonic acid) (HEPES) buffer (100 mM, pH=8.5) and then an aqueous solution of 2′-(4-ethoxyphenyl)-5-(4-methyl-1-piperazinyl)-2,5′-bi-1H-benzimidazole trihydrochloride (Hoechst 33342; 0.5 mg in 250 microliters of DI-water) was added. To the mixture, 5 mg an aqueous solution of 1-ethyl-3-(3-dimethylaminopropyl) carbodiimide (EDC) (5 mg EDC in 50 microliters DI-water) was added. The mixture was then incubated at 32° C. for 15 min and then kept at room temperature (RT) for 15 hrs with mild shaking on a slow rocker (˜50 rpm). The buffer of the mixture was then replaced using two cycles of centrifugal filtration. Each cycle was performed by diluting with phosphate buffered saline (PBS, pH=7.1) to 4 mL and then transferred into Amicon® Ultra-4 centrifugal filter devices (30 kD cut-off) and spun at 2800 rcf for 30 min at RT. The final residue, having the QD-fluorescent dye conjugate nanoparticles, was re-dispersed in 200 microliters of phosphate buffered saline (PBS) and stored at 4° C. until characterization.

### Example 4: Determination of Amount of Fluorescent Dye Conjugated on QD Nanoparticles

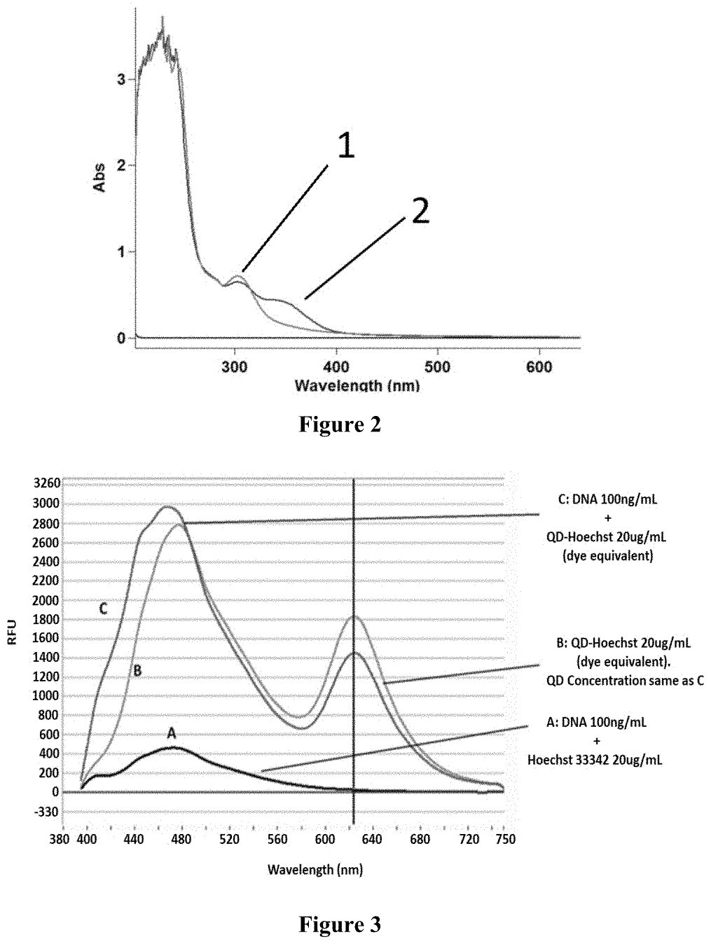

The conjugation of fluorescent dye (Hoechst 33342) to the QD nanoparticles was evaluated by comparing the ultraviolet-visible (UV-Vis) absorption spectra at 350 nm of two QD solutions with exact concentration of QDs. The first QD solution contained QD nanoparticles as used above prior to conjugation with the fluorescent dye. The second quantum dot solution included the QD-fluorescent dye conjugate nanoparticles prepared above. The QD concentration was the same for both the first and the second QD solutions. In FIG. 2, the red trace (1) corresponds to the QD nanoparticles as used above prior to conjugation with the fluorescent dye and the blue trace (2) corresponds to the QD-fluorescent dye conjugate nanoparticles. As can be observed, the UV-Vis absorption spectrum of the QD-fluorescent dye conjugate nanoparticles exhibits a broad peak at about 350 nm, indicating conjugation of the fluorescent dye with the QD nanoparticles.

### Example 5: Enhancement of Emission

The spectral properties of the QD-fluorescent dye conjugate nanoparticles formed in Example 1 were determined in the presence and absence of DNA. A stock solution of DNA (deoxyribonucleic acid sodium salt from salmon testes, Sigma D1626) was prepared (1 mg DNA/mL in tris/borate/EDTA buffer (TBE, pH=8.3)). Then, 5 mL of a diluted solution of DNA (100 ng DNA/mL in DI water) was prepared from the stock solution. To this diluted DNA solution, 3 μL of Hoechst 33342 dye (at 2 mg/mL) was added to give a final Hoechst concentration of 20 μg/mL. The DNA/Hoescht solution was compared to another solution of QD-fluorescent dye conjugate nanoparticles (Ex. 1) that had the same concentration of the Hoechst 33342 dye (20 μg/mL). The spectral comparison were performed using the Nanodrop 3300 fluorimeter using the UV excitation channel at 365 nm. FIG. 3 shows the difference of the emission intensity and peak height from Hoechst dye at the same DNA concentration (100 ng/mL).

### Example 6: Enhancement of Emission and Response to DNA Concentration

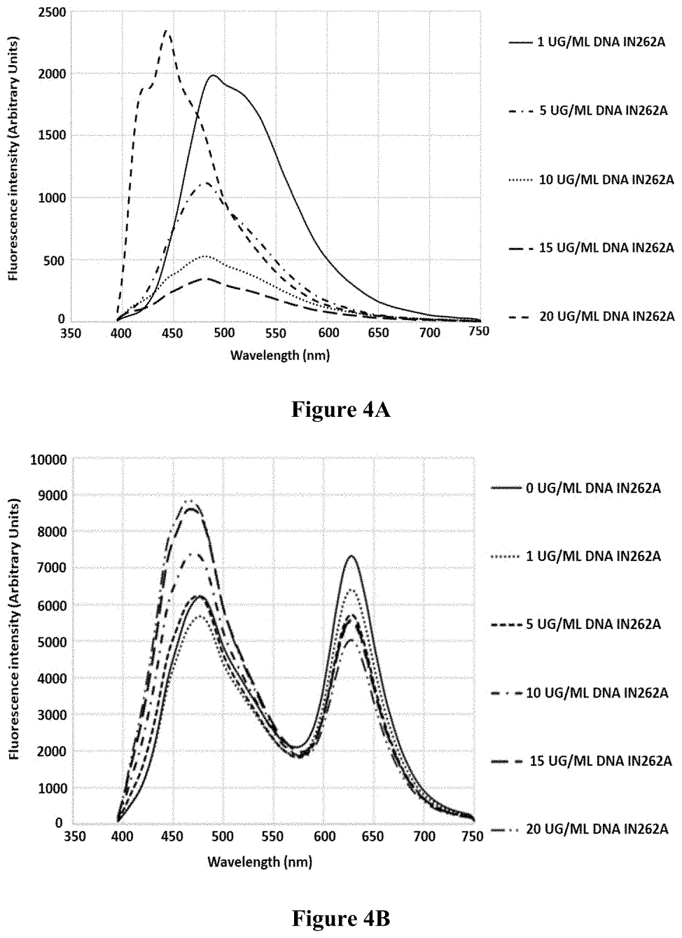

The spectral properties of the QD-fluorescent dye conjugate nanoparticles formed in Example 1 were determined in the presence of different concentrations of DNA. A stock solution of DNA (deoxyribonucleic acid sodium salt from salmon testes, Sigma D1626) was prepared at (1 mg/mL in TBE buffer (pH=8.3)). Then, a series of diluted solutions of DNA was prepared in DI water to give DNA concentrations at 0, 1, 5, 10, 15, and 20 μg/mL. To 300 μL of each diluted solution, 34, of Hoechst 33342 dye (at 2 mg/mL) was added to give a final Hoechst concentration of 20 μg/mL. Each Hoechst 33342 dye-containing solution was then compared to another solution of QD-fluorescent dye conjugate nanoparticles (Ex. 3) at 34 μg/mL (of nanoparticles) to provide an equivalent concentration of Hoechst (20 μg/mL) as measured by UV absorption at 350 nm. The spectral comparison was performed using a Nanodrop 3300 fluorimeter using the UV excitation channel at 365 nm. FIG. 4A shows control 1, 5, 10, 15, and 20 μg/mL DNA and Hoechst 33342 dye-containing solutions. The 1 μg/mL control solution exhibited a luminescence peak at about 487 nm and the 20 μg/mL control solution exhibited a luminescence peak at about 443 nm. The 5, 10, and 15 μg/mL control solutions all exhibited a luminescence peak of about 479 nm. Interestingly, the intensity of luminescence of the 5, 10, and 15 μg/mL control solutions was markedly lower than the 1 and 20 μg/mL control solutions. FIG. 4B shows 0, 1, 5, 10, 15, and 20 μg/mL DNA and QD-Hoechst conjugate-containing solutions. As shown in FIG. 4B below, a strong blue peak at about 465 nm for the QD-Hoechst conjugates are observed. The peak height at 465 nm was responsive to the incremental increase of DNA concentration in the range from 1-20 μg/mL. In each sample shown in FIG. 4B, an emission peak around 630 nm, corresponding to emission from the QD of the QD-Hoechst conjugate, is observed. In all cases, the control solutions, not containing QDs, showed weaker emission peaks that was, with the exception of the 1 μg/mL control solution, reversibly correlated with DNA concentration.

### Example 7: Enhancement of Emission Observed on a Glass Slide and Fluorescence Microscopy

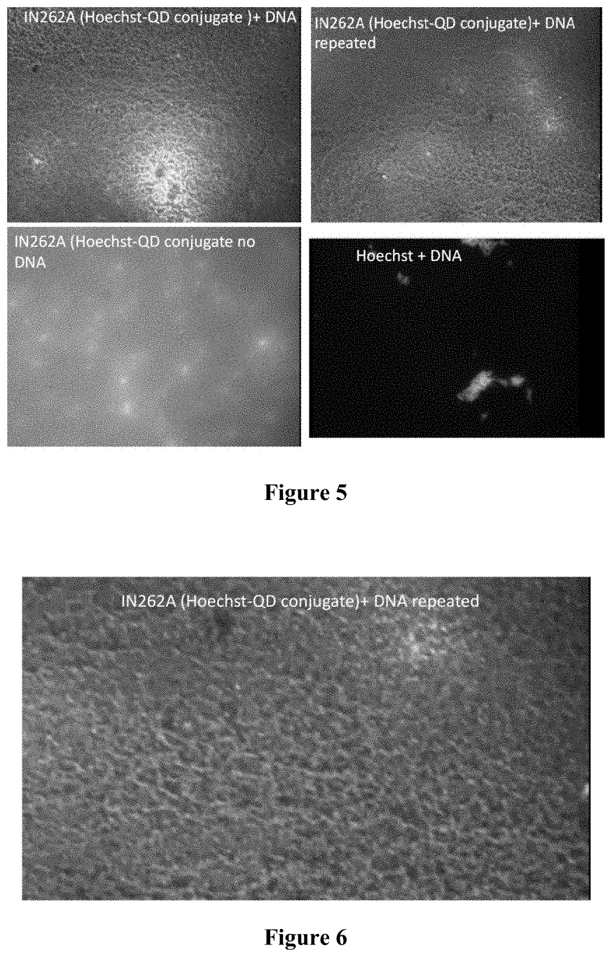

The DNA binding and enhanced detection ability were also observed using trace amounts of DNA mounted on a glass slide. In this experiment, a DNA solution was prepared in DI water at 0.3 μg/mL and was mixed with the QD-fluorescent dye conjugate nanoparticles (Ex. 1) or with the Hoechst 33342 only (control experiment) at a final dye concentration of 0.4 mg/mL. A 3 μL solution of each composition was mounted on a slide, smeared and let to dry. The slide was then observed under a fluorescence microscope (Olympus BX51) using 50× objective and a DAPI filter cube (UMWU2) and equipped with an Osram HBO50W/AC L1 Short arc mercury lamp as an excitation source. As observable from the top two images in FIG. 5, the QD-fluorescent dye conjugate nanoparticles are able to detect the DNA threads mounted on the glass. When the same exact experiment was repeated using the control dye (Hoechst 33342) almost no signal was observed. Without DNA mounted on the slide, the image was fuzzy and could not show crisp image as observed when DNA was present, indicating that the QD-fluorescent dye conjugate nanoparticles were binding and accumulating on the DNA threads, giving the crisp images observed in the top panels. All the images were taken using the same microscopy settings. FIG. 6 indicates an enhanced ability to detect DNA on a glass slide (zoomed in to show DNA strands covered with Hoechst-QDs).

### Example 8: Labelling of Chromosomes, DNA and Nuclear Matter in Live Cells

Cultured cells (approximately 3×10⁶ Mia Pa Ca-2 human pancreatic carcinoma cells in Dulbecco's Modified Eagle's Medium (DMEM)) in a Nunc™ T75 flask were treated with 25 μg/mL in 12 mL culture media QD-fluorescent dye conjugate nanoparticles (Ex. 1) or with plain Hoechst 33342 at 0.1 μg/mL of culture media (12 mL). After 10 min, Colcemid™ 10 μg/mL solution in Hank's Balanced Salt Solution (Democolcine, Sigma Aldrich product code D1925) was added at final concentration of 0.02 μg/mL of culture media to arrest cell cycle at M phase. The cells were cultivated for additional 90 min and then the chromosomes were harvested using a modified standard protocol for chromosomal spread preparation with ice-cold methanol as a fixative. For the standard protocol, see “Chromosome Preparation From Cultured Cells” by Howe et al. (Journal of Visualized Experiments, 83, e50203, January 2014). The modified standard protocol was as follows:
1) Grow cells according to specific cell culturing conditions. When the cells have reached logarithmic phase (80% confluency), add 10 μl/mL of Colcemid to the cell culture flask. A minimum of 2×10⁶ cells is recommended.
2) Incubate cells at 37° C. in a 5% CO₂ incubator for 45 min. Using a sterile pipette, transfer media from cells into a 15 mL conical tube. Set aside.
3) Gently wash the cells by adding 2 ml of HBSS Buffer into the flask. Swirl buffer and then remove using a pipette. Discard.
4) Add 1 mL of trypsin, ensuring that it covers the entire surface of the flask. Only leave the cells in trypsin for about 2 min. Once the majority of the cells have detached, pipette the media in the conical tube back onto the cells.
5) Transfer the cell suspension in 10 mL aliquots into 15 mL conical tubes. Centrifuge at 200×g for 10 min. Remove supernatant and resuspend the pellet.
6) Add 10 mL of 0.075 M KCl which has been pre-warmed to 37° C. to the remaining pellet in the conical tube. Vortex tube at medium speed to mix KCl and cells.
7) Incubate cells at 37° C. for 10 min. Centrifuge at 200×g for 5 min at 25° C. Remove supernatant (until about 0.5 mL remains) and resuspend pellet.
8) Carefully add 5 mL of ice-cold formaldehyde (3.7% in water) to the cells while vortexing. Then add 5 mL more of cold formaldehyde solution without vortexing for a total of 10 mL. Incubate for 1 hr on ice.
9) Centrifuge at 200×g for 5 min at 4° C. Remove supernatant and resuspend cells in ice-cold MeOH. Add 5 mL of cold MeOH to each tube.
10) Centrifuge at 200×g for 5 min at 4° C. Remove supernatant and resuspend cells in ice-cold MeOH. Disperse final pellet in small volume of ice-cold MeOH, drop chromosomes on glass slides, and leave to dry at RT for >2 h to overnight. Use glycerol:water (50:50) as a mounting media.

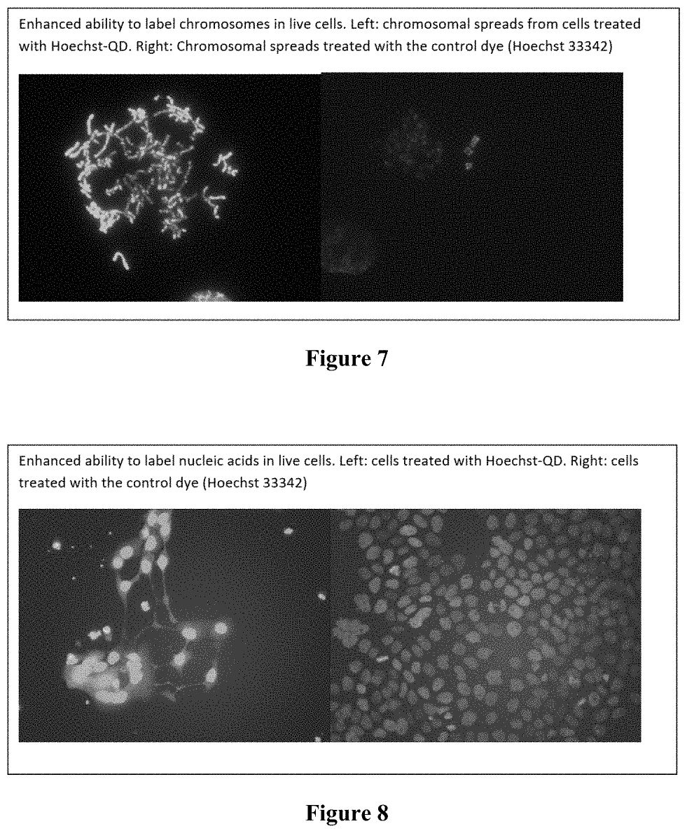

As shown in FIG. 7, the cells treated with QD-fluorescent dye conjugate nanoparticles (left panel) showed very strong labelling when imaged using fluorescent microscopy as compared to the Hoechst 33342 dye alone (right panel).

### Example 9: Labelling of Nuclear and Cytoplasmic DNA or RNA in Live Cells

Cultured cells in Nunc™ T75 flasks (approximately 10×10⁶ cells of A431 human squamous cell carcinoma in Dulbecco's Modified Eagle's Medium (DMEM)) were treated with 50 μg/mL (total volume of culture medium is 12 mL) QD-fluorescent dye conjugate nanoparticles (Ex. 1) or with plain Hoechst 33342 (2 mg/mL stock solution in DI water at a final concentration of 0.2 μg/mL in culture medium (12 mL) for 15h (overnight), then the cells were imaged using fluorescence microscopy as in the previous examples. It is clear from FIG. 8 that the cells treated with the QD-fluorescent dye conjugate nanoparticles are strongly labelled to the extent that the cytoplasmic DNA (or RNA) are also stained. The cell-cell interaction is clearly captured in the left panel unlike the case of the control Hoechst 33342 dye, where the nuclei were stained but without the ability to detect cell-cell communication.

### Example 10: Vectorization of QDs into Nuclei in Live Cells

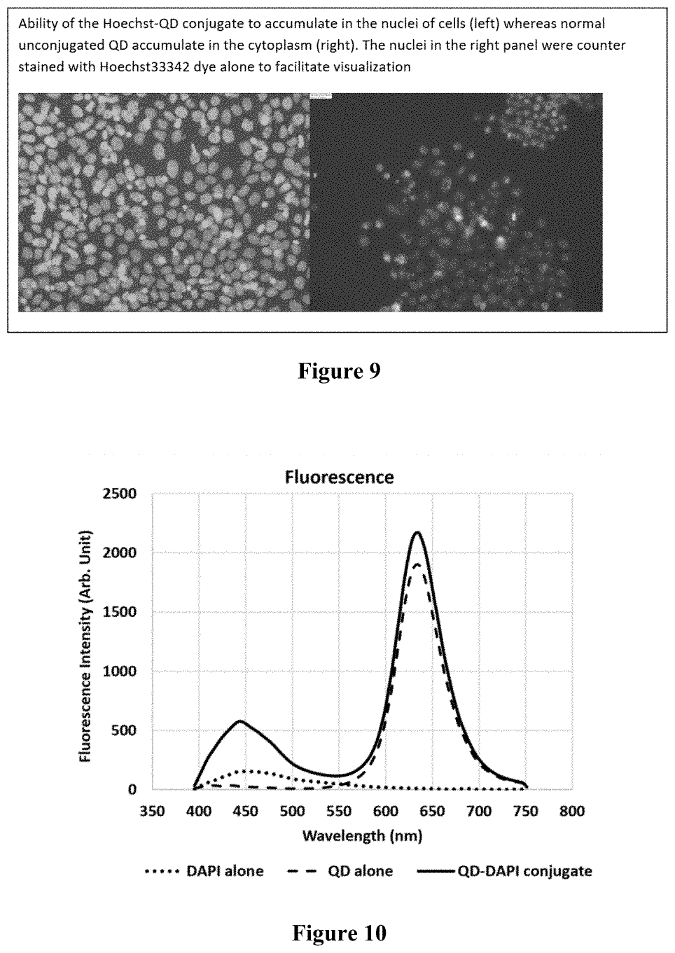

Cultured cells in Nunc T75 flasks containing 12 mL of Dulbecco's Modified Eagle's Medium (DMEM) (MiaPaca2 pancreatic carcinoma or SKB3 human breast carcinoma) were treated at 50 μg/mL final concentration QD-fluorescent dye conjugate nanoparticles (Ex. 1) or with plain QDs at 50 μg/mL for 15h (overnight), then the cells were imaged using fluorescence microscopy as in the previous examples. It is clear from FIG. 9 that the QD-fluorescent dye conjugate nanoparticles accumulate mainly in the nuclei of cells whereas the plain untargeted QD accumulate in the cytoplasmic space, following the typical intracellular distribution path of standard nanoparticles.

### Example 11: Preparation of Nanodiamond-Fluorescent Dye Conjugate Nanoparticles

Five milligrams of carboxylate-functionalized nanodiamonds (Sigma Aldrich catalog No. 901800, 5 nm avg. part. size (DLS), 10 mg/mL in H₂O), are dispersed in 1 mL of 4-(2-hydroxyethyl)-1-piperazineethanesulfonic acid) (HEPES) buffer (100 mM, pH=8.5) and then an aqueous solution of 2′-(4-ethoxyphenyl)-5-(4-methyl-1-piperazinyl)-2,5′-bi-1H-benzimidazole trihydrochloride (Hoechst 33342; 0.5 mg in 250 microliters of DI-water) is added. To the mixture, 5 mg of an aqueous solution of 1-ethyl-3-(3-dimethylaminopropyl) carbodiimide (EDC) (5 mg EDC in 50 microliters DI-water) is added. The mixture is then incubated at 32° C. for 15 min and then kept at room temperature (RT) for 15 hrs with mild shaking on a slow rocker (˜50 rpm). The buffer of the mixture is then replaced using two cycles of centrifugal filtration. Each cycle is performed by diluting with phosphate buffered saline (PBS, pH=7.1) to 4 mL and then transferring into Amicon® Ultra-4 centrifugal filter devices (30 kD cut-off) and spinning at 2800 rcf for 30 min at RT. The final residue, having the nanodiamond-fluorescent dye conjugate, is re-dispersed in 500 microliters of PBS and stored at 4° C. until characterization.

### Example 12: Preparation of Fluorescent Polymer Nanoparticle-Fluorescent Dye Conjugate Nanoparticles

Five milligrams of carboxylate-functionalized fluorescent polymer nanoparticles dispersed in water (Sigma Aldrich catalog No. 904996, iron oxide incorporated conjugated polymer nanoparticles, fluorescence λₑₘ 680 nm, 100m/mL in H₂O) are dispersed in 1 mL of 4-(2-hydroxyethyl)-1-piperazineethanesulfonic acid) (HEPES) buffer (100 mM, pH=8.5) and then an aqueous solution of 2′-(4-ethoxyphenyl)-5-(4-methyl-1-piperazinyl)-2,5′-bi-1H-benzimidazole trihydrochloride (Hoechst 33342; 0.5 mg in 250 microliters of DI-water) is added. To the mixture, 5 mg of an aqueous solution of 1-ethyl-3-(3-dimethylaminopropyl) carbodiimide (EDC) (5 mg EDC in 50 microliters DI-water) is added. The mixture is then incubated at 32° C. for 15 min and then kept at room temperature (RT) for 15 hrs with mild shaking on a slow rocker (˜50 rpm). The buffer of the mixture is then replaced using two cycles of centrifugal filtration. Each cycle is performed by diluting with phosphate buffered saline (PBS, pH=7.1) to 4 mL and then transferring into Amicon® Ultra-4 centrifugal filter devices (30 kD cut-off) and spinning at 2800 rcf for 30 min at RT. The final residue, having the fluorescent polymer nanoparticle-fluorescent dye conjugate, is re-dispersed in 500 microliters of PBS and stored at 4° C. until characterization.

### Example 13: Preparation of Fluorescent Europium Chelate Polymer Nanoparticle-Fluorescent Dye Conjugate Nanoparticles

Five milligrams of carboxylate-functionalized fluorescent europium chelate polymer nanoparticles dispersed in water (Bangs Laboratories, Inc., Catalog No. FCEU001; 0.10 μm europium chelate) is dispersed in 1 mL of 4-(2-hydroxyethyl)-1-piperazineethanesulfonic acid) (HEPES) buffer (100 mM, pH=8.5) and then an aqueous solution of 2′-(4-ethoxyphenyl)-5-(4-methyl-1-piperazinyl)-2,5′-bi-1H-benzimidazole trihydrochloride (Hoechst 33342; 0.5 mg in 250 microliters of DI-water) is added. To the mixture, 5 mg of an aqueous solution of 1-ethyl-3-(3-dimethylaminopropyl) carbodiimide (EDC) (5 mg EDC in 50 microliters DI-water) is added. The mixture is then incubated at 32° C. for 15 min and then kept at room temperature (RT) for 15 hrs with mild shaking on a slow rocker (˜50 rpm). The buffer of the mixture is then replaced using two cycles of centrifugal filtration. Each cycle is performed by diluting with phosphate buffered saline (PBS, pH=7.1) to 4 mL and then transferring into Amicon® Ultra-4 centrifugal filter devices (30 kD cut-off) and spinning at 2800 rcf for 30 min at RT. The final residue, having the fluorescent europium chelate polymer nanoparticle-fluorescent dye conjugate, is re-dispersed in 500 microliters of PBS and stored at 4° C. until characterization.

### Example 14: Preparation of QD-Fluorescent Dye (DAPI) Conjugate Nanoparticles Via Covalent Bonding

Water soluble surface modified quantum dot nanoparticles (5 mg), prepared substantially as described in Example 2 are dispersed in (4-(2-hydroxyethyl)-1-piperazineethanesulfonic acid) (HEPES) buffer (100 mM, pH=8.5) and then an aqueous solution of 4′,6-diamidino-2-phenylindole (DAPI; 0.5 mg in 250 microliters of DI-water) is added. To the mixture, 5 mg of an aqueous solution of 1-ethyl-3-(3-dimethylaminopropyl) carbodiimide (EDC) (5 mg EDC in 50 microliters DI-water) is added. The mixture is then incubated at 32° C. for 15 min and then kept at room temperature (RT) for 15 hrs with mild shaking on a slow rocker (˜50 rpm). The buffer of the mixture is then replaced using two cycles of centrifugal filtration. Each cycle is performed by diluting with phosphate buffered saline (PBS, pH=7.1) to 4 mL and then transferred into Amicon® Ultra-4 centrifugal filter devices (30 kD cut-off) and spun at 2800 rcf for 30 min at RT. The final residue, having the QD-DAPI conjugate nanoparticles, is re-dispersed in 200 microliters of PBS and stored at 4° C. until characterization.

### Example 15: Preparation of QD-Fluorescent Dye (DAPI) Conjugate Nanoparticles Via Physical Adsorption

Water soluble surface modified quantum dot nanoparticles (5 mg), prepared substantially as described in Example 2 were dispersed in deionized water and then an aqueous solution of 4′,6-diamidino-2-phenylindole (DAPI; 0.5 mg in 250 microliters of DI-water) was added. The mixture was then left to stand at room temperature (23° C.) for 1h with mild shaking on a slow rocker (˜50 rpm). The solvent (water) of the mixture was then replaced with phosphate buffered saline (PBS, pH=7.1) using two cycles of centrifugal filtration. Each cycle was performed by diluting with PBS to 4 mL and then transferred into Amicon® Ultra-4 centrifugal filter devices (30 kD cut-off) and spun at 2800 rcf for 30 min at RT. The final residue, having the QD-DAPI conjugate nanoparticles, was re-dispersed in 200 microliters of PBS and stored at 4° C. until characterization.

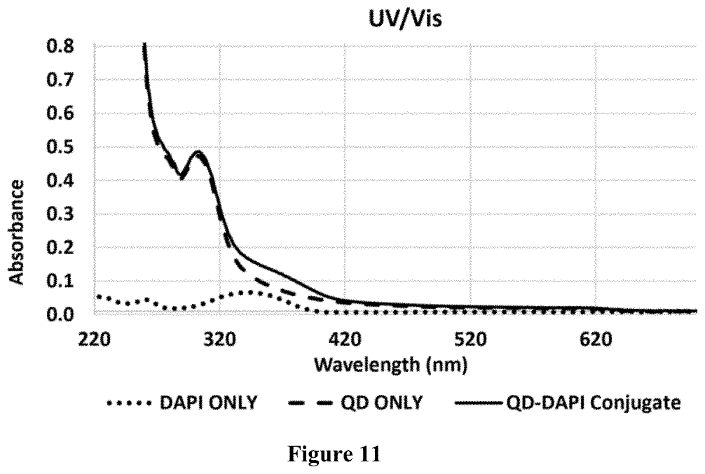

FIG. 10 shows the difference of the fluorescence emission intensity and peak height from DAPI alone, QD alone, and the QD-DAPI conjugate prepared in this example. The corresponding concentrations of both DAPI and QD alone were adjusted to become equal to the concentration of the QD and the due in the QD-DAPI conjugate. As can be seen, the QD-DAPI conjugate exhibits to emission peaks that are noticeably more intense than peaks in the same regions corresponding to the QDs or DAPI alone. FIG. 11 shows that the UV/Vis absorption (extinction coefficient) spectra of DAPI alone, QD alone, and a QD-DAPI conjugate prepared in this example. As can be seen the QD-DAPI conjugate exhibits emission in the region 330 nm-400 nm.

These and other advantages of the invention will be apparent to those skilled in the art from the foregoing disclosure. Accordingly, it is to be recognized that changes or modifications may be made to the above-described embodiments without departing from the broad inventive concepts of the invention. It is to be understood that this invention is not limited to the particular embodiments described herein and that various changes and modifications may be made without departing from the scope of the invention as literally and equivalently covered by the following claims.

What is claimed is:

1. A composition of matter comprising:
a nanoparticle-dye conjugate comprising a photoluminescent or fluorescent nanoparticle associated with a fluorescent dye.

2. The composition of claim 1, wherein the nanoparticle comprises a ligand interactive agent associated with a surface of the nanoparticle.

3. The composition of claim 2, wherein the fluorescent dye is covalently bound to the ligand interactive agent.

4. The composition of claim 2, wherein the fluorescent dye is non-covalently bound to the ligand interactive agent.

5. The composition of claim 2, wherein the nanoparticle further comprises a melamine-based linking/crosslinking agent bound to the ligand interactive agent, the fluorescent dye being bound to the melamine-based linking/crosslinking agent.

6. The composition of claim 1, wherein the nanoparticle is any one of a core quantum dot, a core/shell quantum dot or a core/multishell quantum dot.

7. The composition of claim 1, wherein the nanoparticle is any one of a nanodiamond, a fluorescent silica nanobead, a fluorescent polymer nanoparticle, a fluorescent elemental metal nanoparticle, a rare earth-doped nanoparticle, a graphene quantum dot (GQD), a carbon quantum dot (CQD), or a perovskite quantum dot.

8. The composition of claim 1, wherein the fluorescent dye is any one of:
a) a Hoechst dye;
b) 2-(4-Amidinophenyl)-6-indolecarbamidine dihydrochloride (DAPI);
c) Gallocyanin;
d) a Rhodamine dye;
e) Hematoxylin;
f) a Coumarin dye;
g) a Nile Blue dye;
h) Nile Red;
i) Methylene Blue;
j) an acridine dye;
k) a fluorescein dye;
l) a derivative of a dye according to any one of a)-k); and
m) an analogue of a dye according to any one of a)-k).

9. The composition of claim 1, further comprising an aqueous medium within which the nanoparticle-dye conjugate is dispersed.

10. The composition of claim 9, wherein the aqueous medium is a buffer solution.

11. The composition of claim 1, further comprising a solution comprising DNA or RNA.

12. The composition of claim 1, further comprising cultured cells.

13. The composition of claim 12, wherein the cultured cells are carcinoma cells.

14. A method of imaging and/or labelling a biological sample, the method comprising:
treating a biological sample with a composition of matter according to claim 1; and
associating the nanoparticle-dye conjugate with the biological sample; and
optionally, imaging the nanoparticle-dye conjugate associated biological sample.

15. The method of claim 14, wherein associating the nanoparticle-dye conjugate with the biological sample comprises binding the nanoparticle-dye conjugate with the biological sample.

16. The method of claim 14, wherein associating the nanoparticle-dye conjugate with the biological sample comprises accumulating the nanoparticle-dye conjugate with the biological sample.

17. The method of claim 14, wherein imaging the nanoparticle-dye conjugate associated biological sample comprises fluorescence microscopy.

18. The method of claim 14, wherein the biological sample is any one of a carcinoma cell, a eukaryote, a prokaryote, a nucleic acid, an oligonucleotide, a chromosome, or a spermatozoa.

19. The method of claim 14, further comprising subjecting the nanoparticle-dye conjugate associated biological sample to fluorescence activated cell sorting.

20. The method of claim 14, wherein the method is performed in vivo.
